(19) United States
(12) Patent Application Publication
(10) Pub. No.: US 2011/0124685 A1
(43) Pub. Date: May 26, 2011

(54) Anesthetic Compounds

(76) Inventor: Thomas E. Jenkins, San Carlos, CA (US)

(21) Appl. No.: 12/812,231

(22) PCT Filed: Jan. 13, 2009
(86) PCT No.: PCT/US09/00223

§ 371 Date: Nov. 10, 2010

Related U.S. Application Data
(60) Provisional application No. 61/022,106, filed on Jan. 18, 2008.

(51) Int. Cl. A61K 31/4453; A61P 23/00; C07C 69/92; A61K 31/24; C07D 211/22

(52) U.S. Cl. 514/317; 560/70; 514/534; 546/240

(57) ABSTRACT

The invention provides novel phenolic ester local anesthetic compounds. The invention also provides pharmaceutical compositions that include such compounds as well as methods for making such compounds and compositions and methods for using such compounds and compositions to induce and/or maintain anesthesia and/or analgesia.

Local anesthetics produce loss of sensation by binding to sodium channels and inhibiting sodium currents which causes blockade of sodium channel dependent impulse conduction. The action of local anesthetics is reversible at clinically relevant concentrations, thus allowing for complete recovery of nerve and muscle function without damage to nerve fibers or cells.

Many clinically useful local anesthetics are comprised of a substituted aromatic group attached to a carboxylic acid derivative such as an amide or ester to which is connected a secondary or tertiary amino group via an alkyl linker. Ester anesthetics, whose clinical use was first discovered at the beginning of the 20^{th} century, include, for example, cocaine, procaine, tetracaine, benzocaine, amethocaine and chloroprocaine. Amide anesthetics, which were first clinically used prior to the Second World War, include, for example, lidocaine, prilocaine, mepivacaine, ropivacaine, etidocaine, levobupivacaine and bupivacaine. Despite being discovered first, the use of ester anesthetics has been largely supplanted by amide anesthetics.

Notwithstanding the chemical similarities between these two classes of local anesthetics, clinically important differences exist between amide and ester anesthetics. Significantly, all local anesthetics share similar toxicity profiles (e.g., seizures from central nervous system toxicity, arrhythmia and death from cardiac toxicity), which suggests that the rate of metabolism may be an important factor in fatalities caused by anesthetics. Ester anesthetics are rapidly hydrolyzed in vivo by plasma cholinesterases, while amide anesthetics are hydrolyzed much less rapidly by hepatic proteases. Rapid metabolism prevents esters from reaching toxic level in vivo even with large or repeated doses. Amide anesthetics, in contrast, can accumulate to toxic levels with large or repeated dosages because of slow hydrolysis in vivo.

Another important issue with currently used local anesthetics is the limited duration of action, which is often too short to relieve post-operative pain, and slow onset of action, which limits utility in postoperative settings in addition to aforementioned safety issues. Further, many currently used anesthetics cause pain and discomfort when administered to a patient.

Accordingly, in view of the foregoing, what are needed are local anesthetics which have rapid onset, longer duration of action, and/or minimal side effects.

The present invention satisfies these and other needs by providing novel ester local anesthetics. Also disclosed herein are methods of making novel ester local anesthetics, pharmaceutical compositions of novel ester local anesthetics and methods of using novel ester local anesthetics and pharmaceutical compositions thereof to induce and/or maintain anesthesia and/or analgesia.

In one embodiment the invention provides a compound of the invention that is of formula:

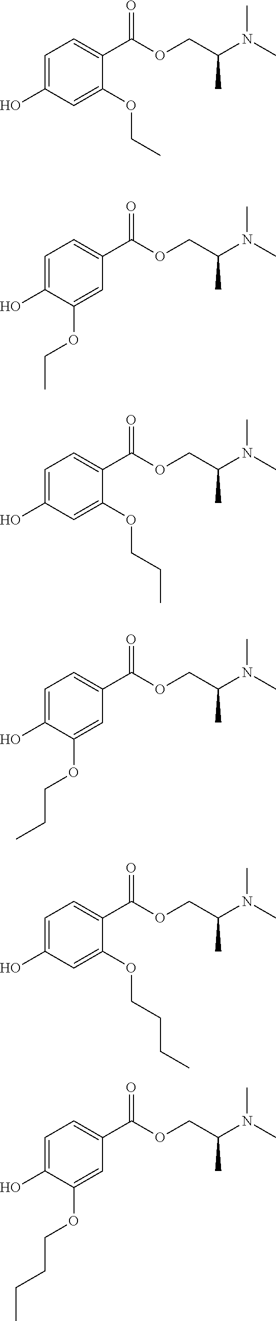

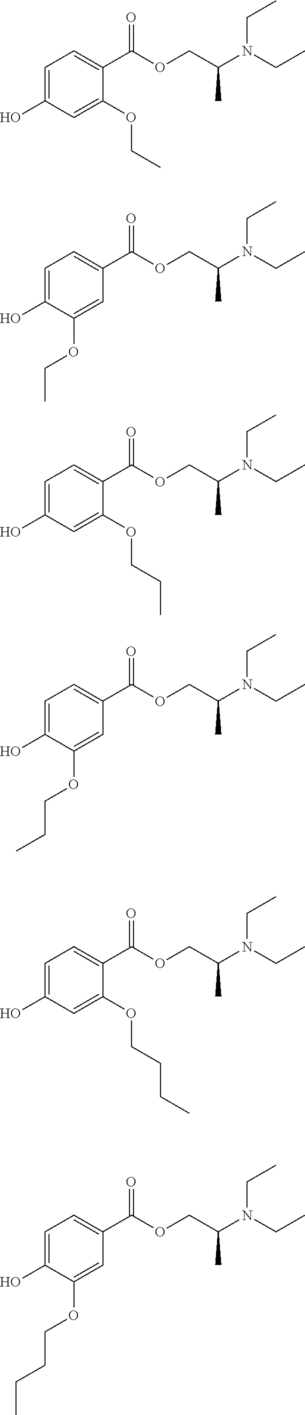

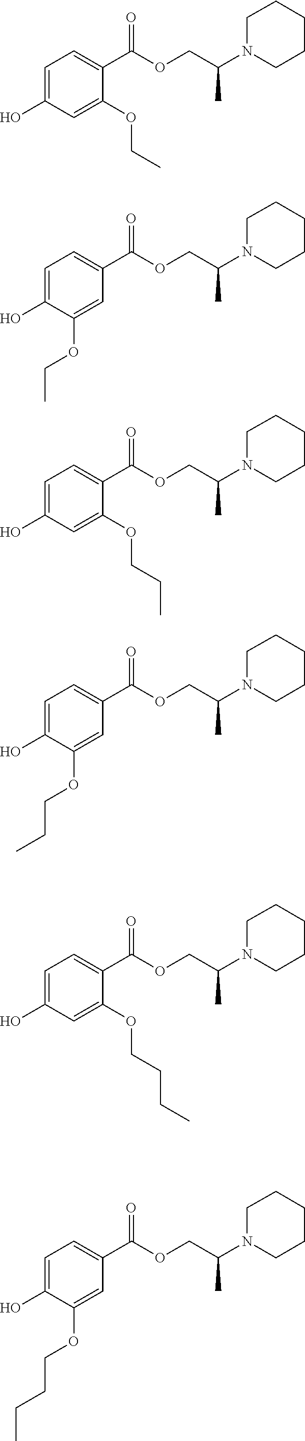

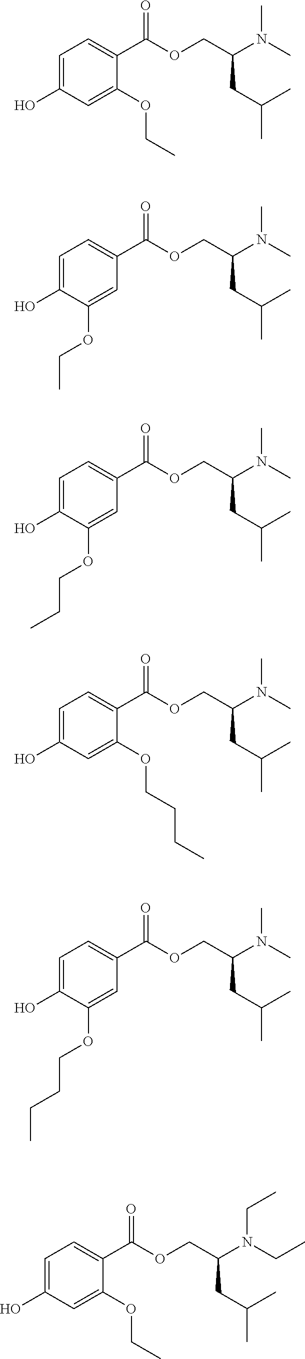

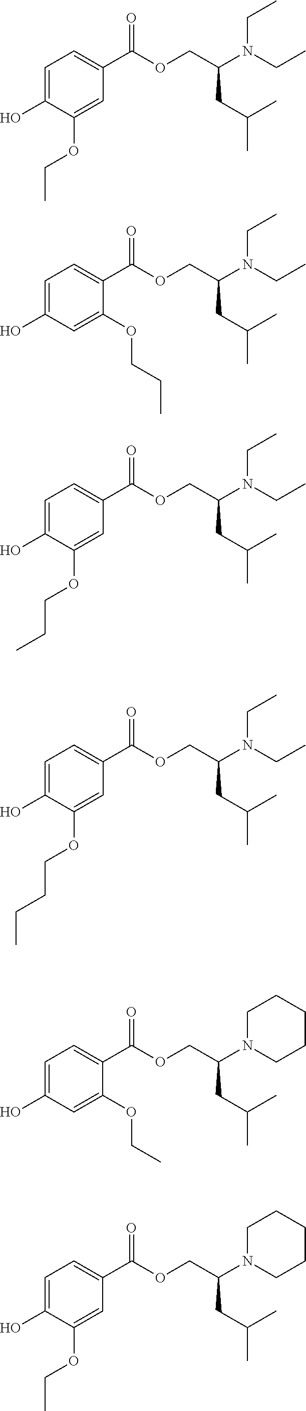

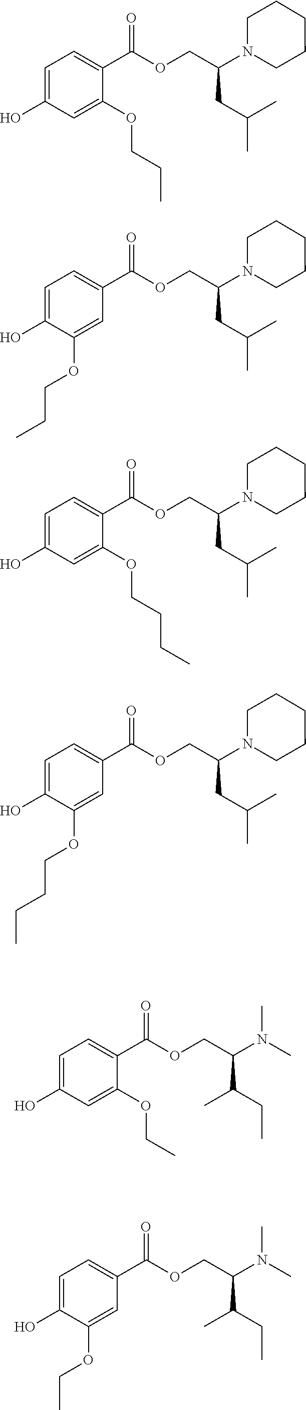

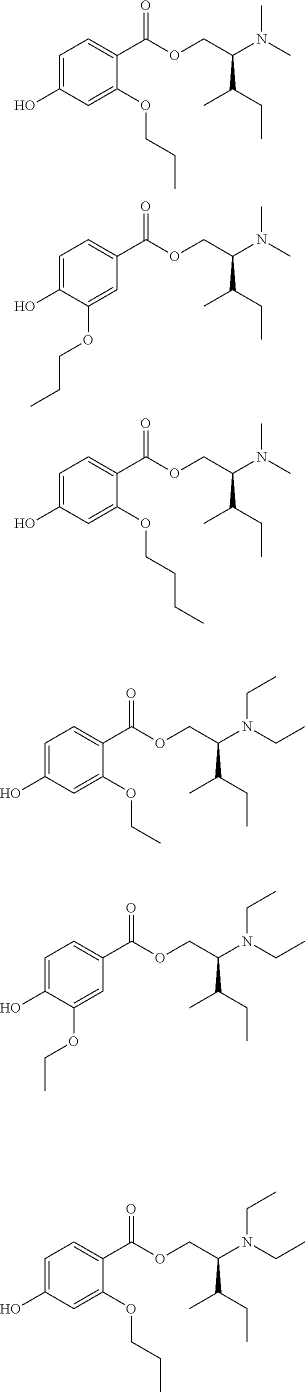

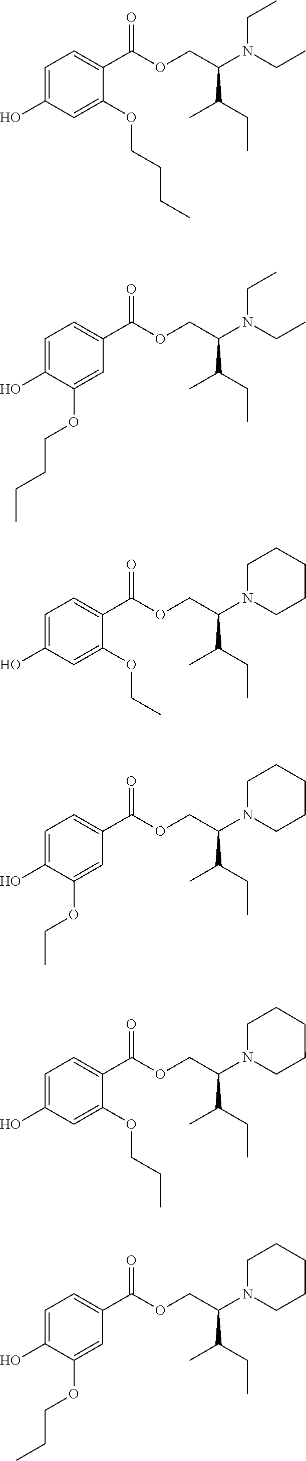

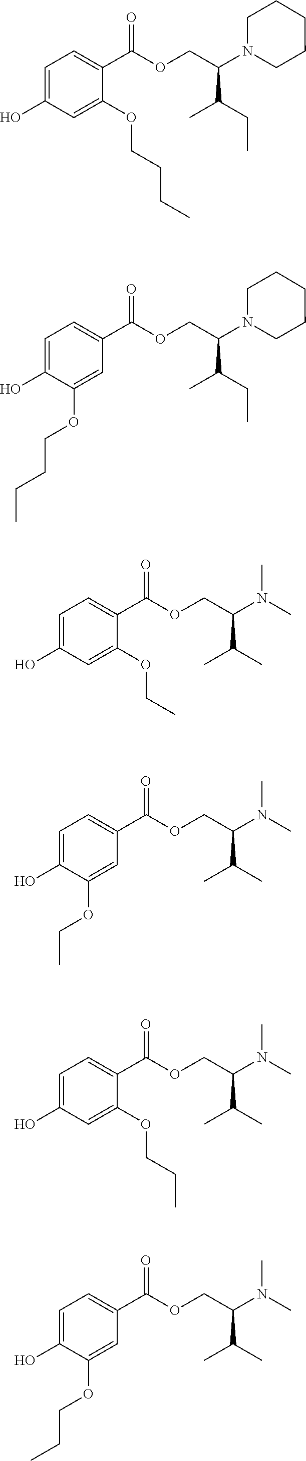

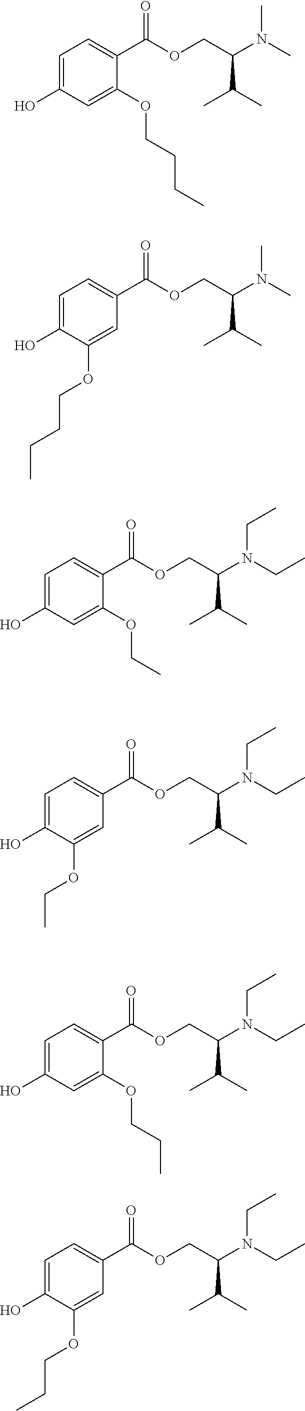

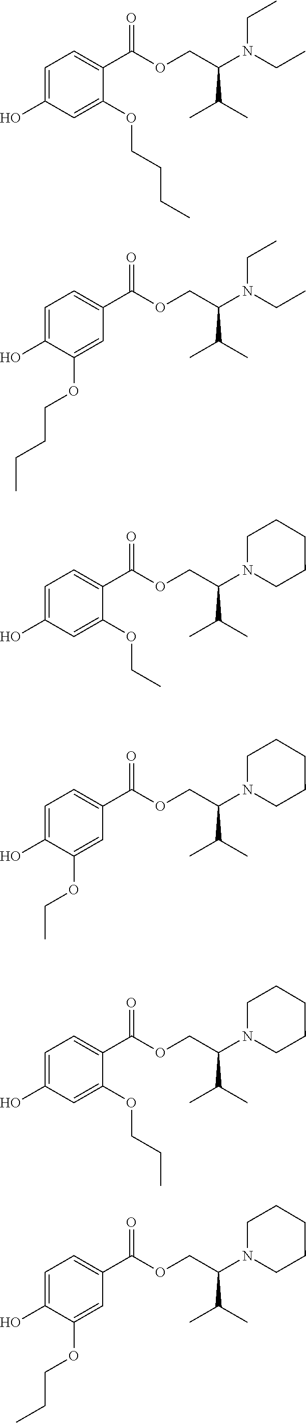

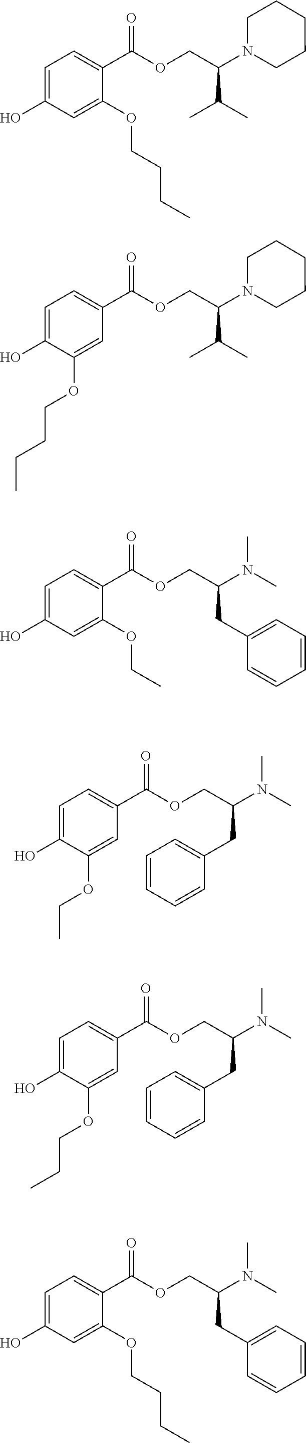

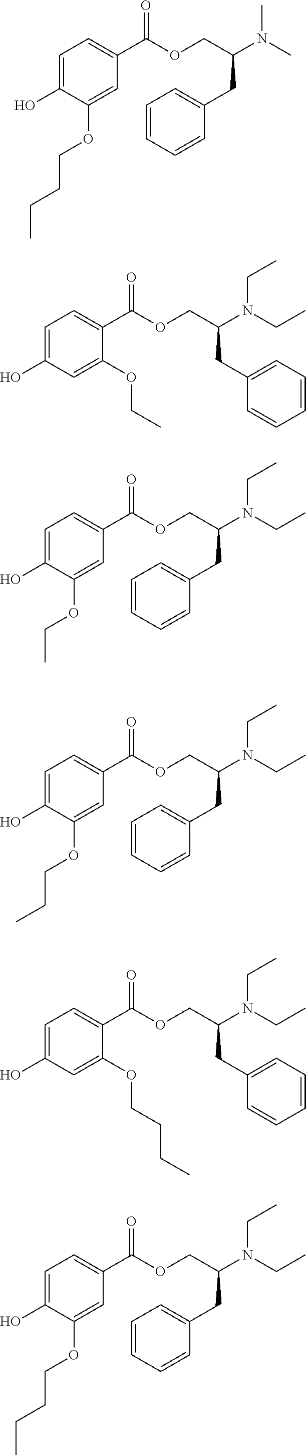

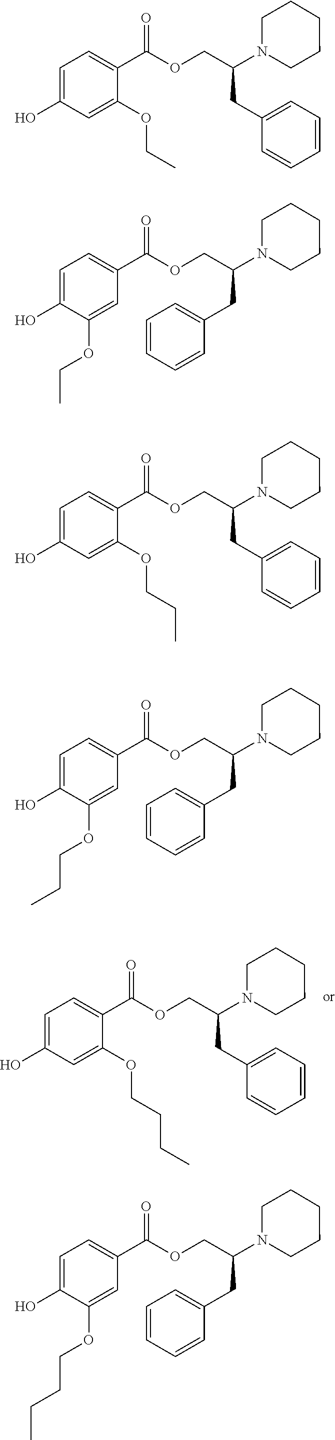

or a prodrug, salt, hydrate, solvate or N-oxide thereof.

In another embodiment the invention provides a method for inducing and/or maintaining anesthesia and/or analgesia comprising administering to a patient in need of such treatment or prevention a therapeutically effective amount of a compound of the invention.

In another embodiment the invention provides a pharmaceutical composition comprising a compound of the invention, and a pharmaceutically acceptable vehicle.

In another embodiment the invention provides a method of inducing or maintaining local anesthesia in a patient comprising administering to the patient a compound of the invention.

In another embodiment the invention provides a method of treating or preventing pain in a patient comprising administering to the patient in need thereof a compound of the invention.

In another embodiment the invention provides a compound of the invention for use in medical therapy.

In another embodiment the invention provides the use of a compound of the invention to prepare a medicament for inducing or maintaining local anesthesia in a mammal such as a human.

In another embodiment the invention provides the use of a compound of the invention to prepare a medicament for treating or preventing pain in a mammal such as a human.

The invention also provides novel processes and synthetic intermediates disclosed herein that are useful for preparing a compound of the invention.

### DETAILED DESCRIPTION

The compounds of the invention are phenolic esters, or prodrugs, salts, hydrates, solvates or N-oxides thereof. Such phenolic esters are advantageous as not only do they effect rapid onset and long duration of action but they also exhibit minimal side effects. For example, compounds of the invention are less likely to induce methemoglobinemia than existing ester-based local anesthetics. Compounds of the invention can be rapidly metabolized via direct conjugative hepatic processes including sulfonation and glucoronidation conjugation, with the resulting conjugates being pharmacologically inactive and subject to rapid renal clearance.

The compounds described herein may contain one or more chiral centers and/or double bonds and therefore, may exist as stereoisomers, such as double-bond isomers (i.e., geometric isomers), enantiomers or diastereomers. Accordingly, all possible enantiomers and stereoisomers of the compounds including the stereoisomerically pure form (e.g., geometrically pure, enantiomerically pure or diastereomerically pure) and enantiomeric and stereoisomeric mixtures are included in the description of the compounds herein. Enantiomeric and stereoisomeric mixtures can be resolved into their component enantiomers or stereoisomers using separation techniques or chiral synthesis techniques well known to the skilled artisan. The compounds may also exist in several tautomeric forms including the enol form, the keto form and mixtures thereof. Accordingly, the chemical structures depicted herein encompass all possible tautomeric forms of the illustrated compounds.

“Pharmaceutical composition” refers to at least one compound and a pharmaceutically acceptable vehicle.

“Pharmaceutically acceptable salt” refers to a salt of a compound, which possesses the desired pharmacological activity of the parent compound. Such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound is replaced by a metal ion, e.g., an alkali metal ion, an alkaline earth ion, or an aluminum ion; or coordinates with an organic base such as ethanolamine, diethanolamine, triethanolamine, N-methylglucamine and the like.

“Pharmaceutically acceptable vehicle” refers to a diluent, adjuvant, excipient or carrier with which a compound is administered.

“Patient” includes mammals, such as humans, livestock, zoo animals and companion animals.

“Therapeutically effective amount” means the amount of a compound that, when administered to a patient for inducing and/or maintaining anesthesia or for providing analgesia, is sufficient to effect such induction or maintenance of anesthesia and/or analgesia. The “therapeutically effective amount” will vary depending on the compound, the disease and its severity, and the age, weight, etc., of the patient to be treated.

### Methods of Synthesis

The compounds of the invention can be prepared as described in the Example. The compounds may also be prepared by other procedures known to those of skill in the art (See e.g., Green et al., “Protective Groups in Organic Chemistry,” (Wiley, 2^{nd} ed. 1991); Harrison et al., “Compendium of Synthetic Organic Methods,” Vols. 1-8 (John Wiley and Sons, 1971-1996); “Beilstein Handbook of Organic Chemistry,” Beilstein Institute of Organic Chemistry, Frankfurt, Germany; Feiser et al., “Reagents for Organic Synthesis,” Volumes 1-17, (Wiley Interscience); Trost et al., “Comprehensive Organic Synthesis,” (Pergamon Press, 1991); “Theilheimer's Synthetic Methods of Organic Chemistry,” Volumes 1-45, (Karger, 1991); March, “Advanced Organic Chemistry,” (Wiley Interscience), 1991; Larock “Comprehensive Organic Transformations,” (VCH Publishers, 1989); Paquette, “Encyclopedia of Reagents for Organic Synthesis,” (John Wiley & Sons, 1995), Bodanzsky, “Principles of Peptide Synthesis,” (Springer Verlag, 1984); Bodanzsky, “Practice of Peptide Synthesis,” (Springer Verlag, 1984). Further, starting materials may be obtained from commercial sources or via well-established synthetic procedures, supra.

Selection of appropriate protecting groups, reagents and reaction conditions is well within the ambit of those of skill in the art. Other methods for synthesis of the compounds described herein will be readily apparent to the skilled artisan and may be used to provide the compounds described herein. Accordingly, the methods presented herein are illustrative rather than comprehensive.

### Therapeutic Methods of Use

In general, the compounds disclosed herein or pharmaceutical compositions thereof may be used to induce and/or maintain local anesthesia and analgesia and are particularly useful for the prophylaxis and/or treatment of pain. As local anesthetics, the compounds disclosed herein or pharmaceutical compositions thereof are useful for regional anesthesia, e.g., topical anesthesia, infiltration anesthesia, perisurgical tissue anesthesia, field block anesthesia, peripheral nerve block anesthesia, epidural anesthesia, spinal anesthesia, bier block anesthesia (local anesthetic injection into an extremity isolated by a tourniquet) and combinations thereof. The compounds disclosed herein or pharmaceutical compositions thereof may also be used to relieve or prevent the pain associated with venipuncture, lumbar puncture, myringtomy, arterial cannulation, neuropathic pain, trauma and tissue ischemia. The compounds disclosed herein or pharmaceutical compositions thereof may be applied topically via patches, or other reservoir systems, bandages or gauzes, creams, ointments or other transdermal delivery systems to treat and/or prevent the pain associated with, for example, dermatoses, hemorrhoids and burns.

### Pharmaceutical Compositions

The pharmaceutical compositions disclosed herein comprise a local anesthetic disclosed herein with a suitable amount of a pharmaceutically acceptable vehicle, so as to provide a form for proper administration to a subject.

Suitable pharmaceutical vehicles include excipients such as starch, glucose, lactose, sucrose, gelatin, malt, rice, flour, chalk, silica gel, sodium stearate, glycerol monostearate, talc, sodium chloride, cellulose, hydroxycellulose, lactose, methylcellulose, polyvinylpyrrolidone, microcrystalline cellulose, gum acacia, dried skim milk, glycerol, propylene, glycol, water, ethanol, polyoxyethylene sorbitan derivatives and the like. The present pharmaceutical compositions, if desired, can also contain minor amounts of wetting or emulsifying agents, or pH buffering agents. In addition, auxiliary, stabilizing, thickening, lubricating and coloring agents may be used.

Pharmaceutical compositions may be manufactured by means of conventional mixing, dissolving, granulating, dragee-making, levigating, emulsifying, encapsulating, entrapping or lyophilizing processes. Pharmaceutical compositions may be formulated in any conventional manner using one or more physiologically acceptable carriers, diluents, excipients or auxiliaries, which facilitate processing of compositions and compounds disclosed herein into preparations which can be used pharmaceutically. Proper formulation is dependent upon the route of administration chosen.

The present pharmaceutical compositions can take the form of solutions, suspensions, emulsions, powders, sustained-release formulations, aerosols, sprays, suspensions or any other form suitable for use known to the skilled artisan. Other examples of suitable pharmaceutical vehicles have been described in the art (see Remington's Pharmaceutical Sciences, Philadelphia College of Pharmacy and Science, 19th Edition, 1995).

In still other embodiments, the dosage form comprises compounds disclosed herein coated on a polymer substrate. The polymer can be an erodible, or a nonerodible polymer. The coated substrate may be folded onto itself to provide a bilayer polymer drug dosage form. For example, compounds disclosed herein can be coated onto a polymer such as a polypeptide, collagen, gelatin, polyvinyl alcohol, polyorthoester, polyacetyl, or a polyorthocarbonate and the coated polymer folded onto itself to provide a bilaminated dosage form. In operation, the bioerodible dosage form erodes at a controlled rate to dispense the compounds over a sustained release period. Representative biodegradable polymers comprise a member selected from the group consisting of biodegradable poly(amides), poly(amino acids), poly(esters), poly(lactic acid), poly(glycolic acid), poly(carbohydrate), poly(ortho ester), poly(orthocarbonate), poly(acetyl), poly(anhydrides), biodegradable poly(dihydropyrans), and poly(dioxinones) which are known in the art (Rosoff, Controlled Release of Drugs, Chap. 2, pp. 53-95 (1989); Heller et al., U.S. Pat. No. 3,811,444; Michaels, U.S. Pat. No. 3,962,414; Capozza, U.S. Pat. No. 4,066,747; Schmitt, U.S. Pat. No. 4,070,347; Choi et al., U.S. Pat. No. 4,079,038; Choi et al., U.S. Pat. No. 4,093,709).

In other embodiments, the dosage form comprises compounds disclosed herein loaded into a polymer that releases the drug(s) by diffusion through a polymer, or by flux through pores or by rupture of a polymer matrix. The drug delivery polymeric dosage form comprises a concentration of 10 mg to 2500 mg homogenously contained in or on a polymer. The dosage form comprises at least one exposed surface at the beginning of dose delivery.

The non-exposed surface, when present, is coated with a pharmaceutically acceptable material impermeable to the passage of the drug(s). The dosage form may be manufactured by procedures known in the art. An example of providing a dosage form comprises blending a pharmaceutically acceptable carrier like polyethylene glycol, with a known dose of compositions and/or compounds disclosed herein at an elevated temperature, (e.g., 37° C.), and adding it to a silastic medical grade elastomer with a cross-linking agent, for example, octanoate, followed by casting in a mold. The step is repeated for each optional successive layer. The system is allowed to set for about 1 hour, to provide the dosage form. Representative polymers for manufacturing the dosage form comprise a member selected from the group consisting of olefin, and vinyl polymers, addition polymers, condensation polymers, carbohydrate polymers, and silicone polymers as represented by polyethylene, polypropylene, polyvinyl acetate, polymethylacrylate, polyisobutylmethacrylate, poly alginate, polyamide and polysilicone. The polymers and procedures for manufacturing them have been described in the art (Coleman et al., Polymers 1990, 31, 1187-1231; Roerdink et al., Drug Carrier Systems 1989, 9, 57-10; Leong et al., Adv. Drug Delivery Rev. 1987, 1, 199-233; Roff et al., Handbook of Common Polymers 1971, CRC Press; Chien et al., U.S. Pat. No. 3,992,518).

For topical administration a compound disclosed herein may be formulated as emulsions, solutions, gels, ointments, creams, suspensions, jellies etc. as are well-known in the art.

Systemic formulations include those designed for administration by injection, e.g., subcutaneous, intravenous, intramuscular, intrathecal, infiltration or intraperitoneal injection, as well as those designed for transdermal, transmucosal, oral or pulmonary administration. Systemic formulations may be made in combination with a further active agent that improves mucociliary clearance of airway mucus or reduces mucous viscosity. These active agents include but are not limited to sodium channel blockers, antibiotics, N-acetyl cysteine, homocysteine and phospholipids.

For injection, compounds disclosed herein may be formulated in aqueous solutions, such as physiologically compatible buffers such as Hanks' solution, Ringer's solution, physiological saline buffer or in the form of an emulsion (as a water-in-oil or oil-in-water emulsion). In one embodiment of the invention, the compound is formulated for injection with an organic acid buffer system (e.g. a (C1-C6) organic acid such as citric, succinic, or acetic acid). The solution may also contain formulatory agents such as suspending, stabilizing and/or dispersing agents. Alternatively, compounds disclosed herein may be in powder form for constitution with a suitable vehicle, e.g., sterile pyrogen-free water, before use.

Vasoconstrictors (e.g., epinephrine or phenylepinephrine), corticosteroids (demaxthasone, cortisone, hydrocortisone, prednisone, beclamethasone, betamethasone, flunisolide, methylprednisone, prednisolone, triamcinolone, alcolmetasone, amcinonide, clobestal, fludrocortisone, difluorsone diacetate, fluocinolone acetonide, fluoromethalone, flurandrenolide, halcinonide, medrysone, etc.) and/or permeability enhancers (e.g., sodium cholate, sodium glycocholate, sodium glycodeoxycholate, taurodeoxycholate, sodium deoxycholate, sodium lithiocholate, chenocholate, chenodeoxycholate, ursocholate, ursodeoxycholate, hydrodeoxycholate, dehydrocholate, glycochenolate, taurochenocholate, taurochenodeoxycholate, etc.) can also be added to the present pharmaceutical compositions.

### Therapeutic/Prophylactic Administration and Doses

When used to maintain and/or induce anesthesia and/or analgesia, the compounds disclosed herein and/or pharmaceutical compositions thereof may be administered alone or in combination with other pharmaceutical agents including compounds disclosed herein and/or pharmaceutical compositions thereof. The compounds disclosed herein may be administered or applied per se or as pharmaceutical compositions. The specific pharmaceutical composition depends on the desired mode of administration, as is well known to the skilled artisan.

Compounds disclosed herein and/or pharmaceutical compositions thereof may be administered to a subject by injection including intravenous injection, continuous infusion, intramuscular injection, subcutaneous injection, transdermally, intracerebrally, intravaginally, rectally, topically, particularly to the ears, nose, eyes, or skin or any other convenient method known to those of skill in the art. In some embodiments, compounds disclosed herein and/or pharmaceutical compositions thereof are delivered by infiltration methods such as, for example, peripheral nerve blocks, serosal and neuraxial delivery, (e.g., epidural, caudal, etc.)

In one embodiment, compounds disclosed herein and/or pharmaceutical compositions thereof are administered transdermally. The transdermal delivery of drugs has become a proven technology that offers a variety of significant clinical benefits over alternative routes of administration. Because transdermal drug delivery offers sustained and controlled release of the drug into the patient, it enables a steady blood level to be maintained for an extended period of time. This often results in reduced systemic side effects and, sometimes, improved efficacy over other dosage forms.

The efficiency of drug transport into or through the skin depends on a number of factors such as the condition and type of skin, the physicochemical characteristics of the permeant (the drug), other chemicals present in the dosage form (e.g. penetration enhancers), and external conditions (e.g. temperature). For example, compounds of the invention can be co-formulated or co-delivered with penetration/permeability enhancers, which can be, for example, chemical, thermal and/or physical.

The factors with perhaps the greatest influence are the physicochemical characteristics of the drug molecule. Based on the current understanding of the physicochemical features of molecules that are efficiently transported into and through the skin, it can be concluded that many of the compounds of the invention possess favorable physicochemical characteristics for transdermal penetration. These characteristics include parameters such as Log P (the log of the octanol:water partition coefficient), aqueous solubility of the freebase form, and the molecular weight/molar volume. Accordingly, in one embodiment the invention provides compounds of the invention that possess physicochemical characteristics that are favorable for transdermal penetration.

Transdermal devices can also be used to deliver the compounds disclosed herein and/or pharmaceutical compositions thereof. Transdermal devices can include or be co-formulated or co-delivered with penetration/permeability enhancers. In some embodiments, the transdermal device is a matrix type transdermal device (Miller et al., International Publication No. WO 2004/041324). In other embodiments, the transdermal device is a multi-laminate transdermal device (Miller, United States Patent Application Publication No. 2005/0037059).

The amount of compounds disclosed herein and/or pharmaceutical compositions thereof that will be effective in the treatment or prevention of pain in a patient will depend on the specific nature of the pain condition and can be determined by standard clinical techniques known in the art. The amount of compounds disclosed herein and/or pharmaceutical compositions thereof administered will, of course, be dependent on, among other factors, the subject being treated, the weight of the subject, the severity of the affliction, the manner of administration and the judgment of the prescribing physician.

### Combination Therapy

In certain embodiments, compounds disclosed herein and/or pharmaceutical compositions thereof can be used in combination therapy with other therapeutic agents, such as, for example, other local ester anesthetics and/or local amide anesthetics. The compounds disclosed herein and/or pharmaceutical compositions thereof and the therapeutic agent can act additively or, more preferably, synergistically. In some embodiments, compounds disclosed herein and/or pharmaceutical compositions thereof are administered concurrently with the administration of another therapeutic agent. For example, compounds disclosed herein and/or pharmaceutical compositions thereof may be administered together with another therapeutic agent. In other embodiments, compounds disclosed herein and/or pharmaceutical compositions thereof are administered prior or subsequent to administration of other therapeutic agents.

### Screening

The quality and duration of local anesthetic block produced by a test compound can be evaluated using the following Rat Sciatic Nerve Assay.

### Rat Sciatic Nerve Assay

This assay enables evaluation of the efficacy of local anesthetic molecules via application of the compounds adjacent to the sciatic nerve in the rat and measuring the anesthetic effects on response to pain stimulus (toe pinch), motor function (postural hindlimb thrust) and proprioception (ability of the animal to balance) measured over time.

Each rat is tested for baseline hindpaw motor, proprioceptive, and sensory function and then injected with a test solution over the right sciatic nerve. All tests are performed bilaterally starting on the side that is not injected.

Sciatic nerve injection: Rats are shaved over the right sciatic area. Rats are lightly anesthetized with 1.5% isoflurane until it is possible to manipulate the hip joint. Test Solution(s) (200 uL) are injected proximal to the sciatic nerve using an insulin syringe directed between the greater trochanter and the ischial tuberosity.

Evaluations of anesthetic block include motor, sensory, and proprioceptive block assessed from injection time until all forms of block are absent.

Motor function is evaluated by measuring the extensor postural thrust of the bilateral hind limbs. The rat is held upright over a scale with the hind limbs extended so that the body weight is supported by the distal metatarsus and toes of one hindlimb. The extensor thrust is measured as the gram force applied to the scale as the rat is pushed down until the heel touches the balance. The pretreatment control value is considered to be 0% of the maximal possible effect (MPE). The reduction in this force, representing reduced extensor muscle contraction induced by motor blockade, is calculated as percentage of the control force. A force <20 g is considered to be 100% of the MPE.

Proprioception is evaluated with a balancing test. A hopping response is evoked by lifting the rat into a vertical position with hindlimbs resting on the table and lifting one hindlimb at a time off the table top so that the animal's weight is resting on one hindlimb, then moving the rat laterally until it hops to the side to remain upright. A predominantly motor impairment causes a prompt but weaker than normal response. Conversely, with a predominantly proprioceptive blockade, delayed hopping is followed by greater lateral hops to avoid falling over or, in case of full blockade, no hopping at all. The proprioceptive deficit is compared to the baseline response and graded as 4 (equal to baseline or 0% MPE), 3 (slightly impaired), 2 (moderately impaired), 1 (severely impaired) and 0 (complete or 100% MPE).

Nociceptive responses are measured with a forceps pinch test. The withdrawal response to forceps pinch applied to the distal phalanx of digit 5 is compared to the baseline response and graded as 4 (equal to baseline or 0% MPE), 3 (slightly impaired), 2 (moderately impaired), 1 (severely impaired), and 0 (absent or 100% MPE).

Representative compounds of the invention can be tested in the above Rat Sciatic Nerve Assay. The compounds can demonstrate clinically relevant local anesthetic activity. For example, the efficacy (time of complete conduction block) can be superior to that demonstrated by lidocaine and can be superior to that demonstrated by bupivacaine. Full clinical recovery can be observed in animals tested, demonstrating that the nerve blocks are fully reversible (i.e., the conduction block is likely not due to nerve damage induced by the compounds).

The invention will now be illustrated by the following non-limiting Example.

### Example

Compounds of the invention can be prepared using the following general synthetic scheme.

a. Synthesis of Substituted Amino Alcohol Coupling Pieces:

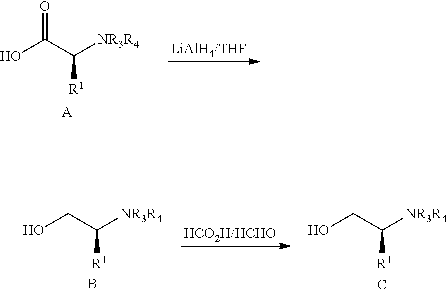

To a solution of a polar non protic solvent such as THF is charged a hydride source, such as lithium aluminum hydride (0.5 to 2.0 equivalents) followed by an amino acid, such as L-leucine, L-isoleucine, L-phenyalanine, L-valine or L-alanine (Compound A), and the mixture is refluxed for 12-24 hours until reduction is complete. This mixture is cooled to 0-10 C, diluted with a polar non protic solvent such as diethyl ether and is quenched with water and basified with a sodium hydroxide solution, such as 15% sodium hydroxide solution. The resulting solid is washed with this polar non-protic solvent, dried with magnesium or sodium sulfate and concentrated in vacuo to afford Compound B as a liquid.

b. Synthesis of Piperidinyl-Containing Amino Alcohol Intermediates

To a solution of Compound B is charged an alcoholic solvent such as ethanol, followed by potassium or sodium carbonate followed by addition of 1,5-dibromopentane and the reaction mixture is heated to reflux for 48-72 hours, then cooled to room temperature and filtered through a bed of celite. Concentration of the organic layer in vacuo gives an oil which is purified by silica gel chromatography. The fractions containing pure product are combined and the solvent removed in vacuo to provide piperidinyl-containing Compound C.

c. Synthesis of Dimethyl-Containing Amino Alcohol Intermediates

To a solution of formic acid and formaldehyde is charged Compound B and the reaction mixture is heated to reflux for 16-24 hours and then is cooled to room temperature at which time a sodium hydroxide solution is charged followed by an organic solvent such as ethyl acetate. After washing of the organic layer with brine and drying with sodium or magnesium sulfate and filtration, the filtrate is concentrated in vacuo to yield dimethyl-containing Compound C.

d. Synthesis of Diethyl-Containing Amino Alcohol Intermediates

To a solution of ethyl iodide, potassium carbonate, and dimethylformamide (DMF) is charged Compound B. The reaction is stirred for 3 hours at room temperature until reaction is determined to be complete by thin layer chromatography (TLC). The reaction mixture is poured into water and extracted with ethyl acetate. After washing of the organic layer with brine and drying with sodium or magnesium sulfate and filtration, the filtrate is concentrated in vacuo to yield diethyl-containing Compound C.

e. Synthesis of Common Intermediate Compound G:

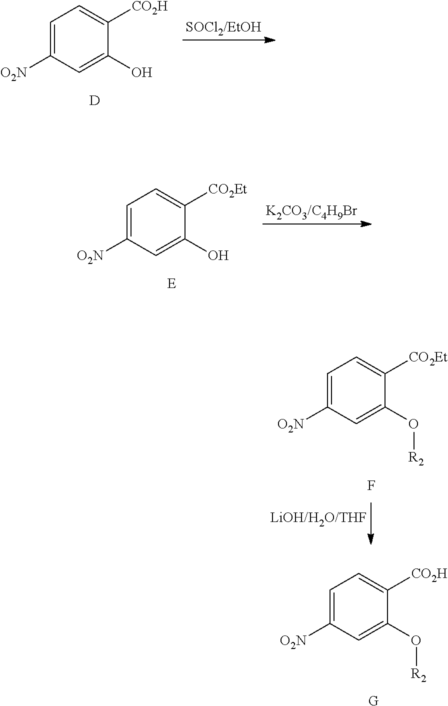

To a solution of a protic polar solvent, such as ethanol, is charged 2-hydroxy-4-nitrobenzoic acid (Compound D) followed by a chlorinating agent such as thionyl chloride. After reflux for 8-16 hours the reaction mixture is concentrated and extracted with an organic solvent such as ethyl acetate, washed with sodium carbonate solution, water and brine and the organic evaporated in vacuo to afford Compound E as a yellow solid.

f. To a solution of a polar non-protic solvent such as DMF is charged Compound E and potassium carbonate followed by an alkyl bromide (such as n-ethyl, n-propyl or n-butyl bromide) and the reaction mixture is stirred at room temperature for 16-30 hours. The reaction mixture is diluted with an organic solvent, such as ethyl acetate, and is washed with water and brine and concentrated in vacuo to afford Compound F as a yellow solid.
g. To a solution of a polar non-protic organic solvent such as THF and water is charged Compound F and an alkyl hydroxide, such as lithium hydroxide, and the solution is stirred at room temperature for 8-16 hours. The reaction mixture is concentrated, acidified with an HCl solution, such as 1.5 M HCl solution and is extracted with an organic solvent such as ethyl acetate. The organic solution is washed with water and brine and dried over magnesium or sodium sulfate and concentrated in vacuo to afford the Compound G as an off-white solid.
h.

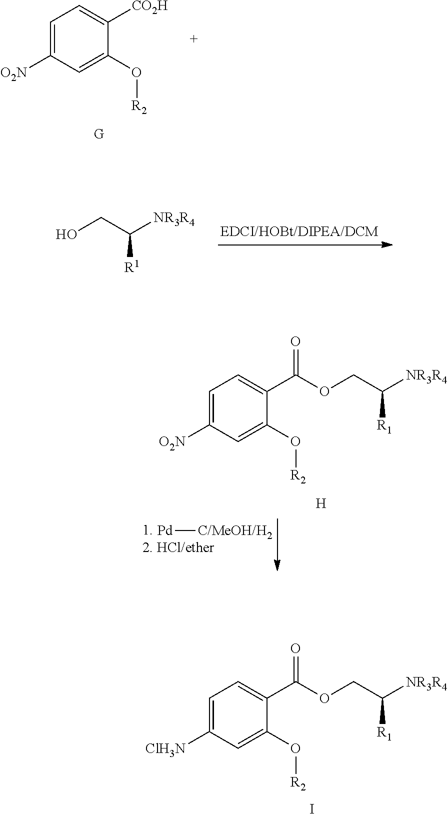

To a solution of a non-polar organic solvent is charged Compound G, an amino alcohol (e.g., piperidinyl-containing Compound C, dimethyl-containing Compound C, or diethyl-containing Compound C), EDCI, HOBt and an amine base, (e.g., Hunig's base), and the reaction mixture is allowed to stir overnight at room temperature. The reaction mixture is then diluted with water, extracted with a non-polar organic solvent and washed with bicarbonate solution, brine and dried with magnesium or sodium sulfate. Purification by silica gel chromatography Compound H as an oil.

i. To a solution of an alcoholic solvent, preferably methanol, is charged Compound H, a Pd—C catalyst and hydrogen gas is introduced and the reaction mixture stirred for several hours. The reaction mixture is filtered over celite, the filtrate is evaporated under reduced pressure and the residue is purified by silica gel chromatography to give the free base of Compound I. The free base is dissolved in a non-polar organic solvent and HCl in a non-polar organic solvent is introduced. The solution is concentrated to dryness and an organic solvent, such as diethyl ether, is charged resulting in precipitation of a solid which is collected to afford Compound I as its hydrochloride salt.
j. It is to be appreciated by one of skill in the art that compounds of the invention having 3-ethoxy, 3-propoxy, or 3-butoxy groups (instead of 2-ethoxy, 3-propoxy, or 3-butoxy groups) can be produced by a similar method (a)-(i), except that Compound D is 3-hydroxy-4-nitrobenzoic acid.

It will be apparent to those skilled in the art that many modifications, both to materials and methods, may be practiced without departing from the scope of this disclosure. Accordingly, the present embodiments are to be considered as illustrative and not restrictive, and the invention is not to be limited to the details given herein, but may be modified within the scope and equivalents of the allowed claims.

All publications and patents cited herein are incorporated by reference in their entirety.

1. A compound of formula 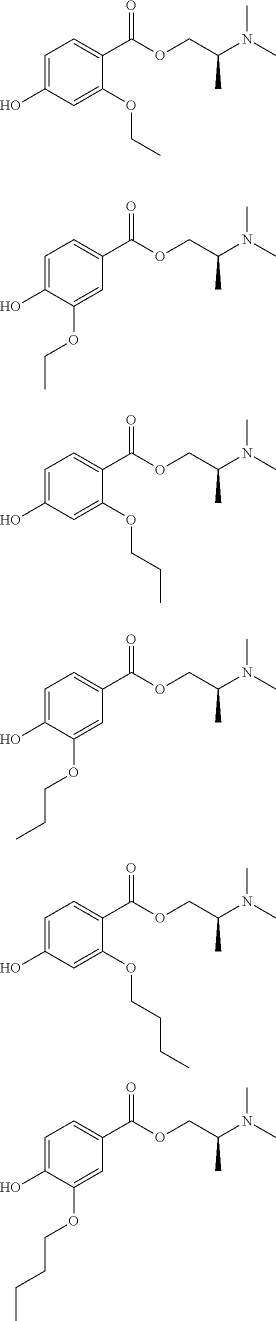 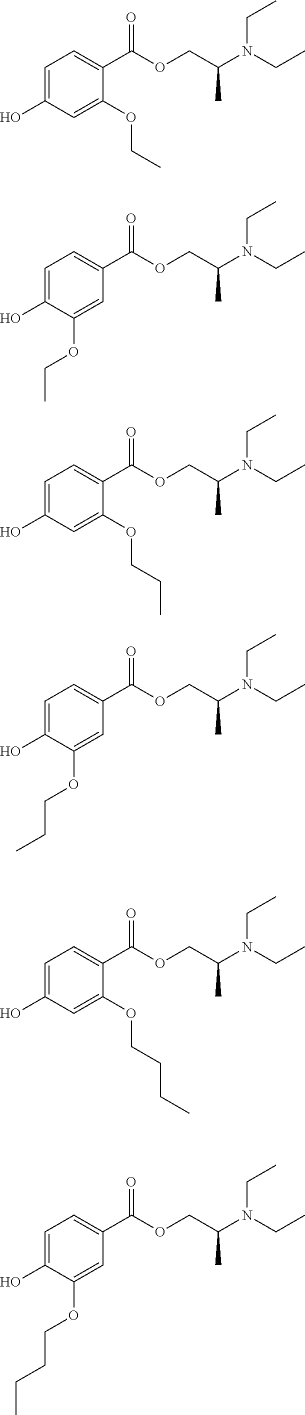 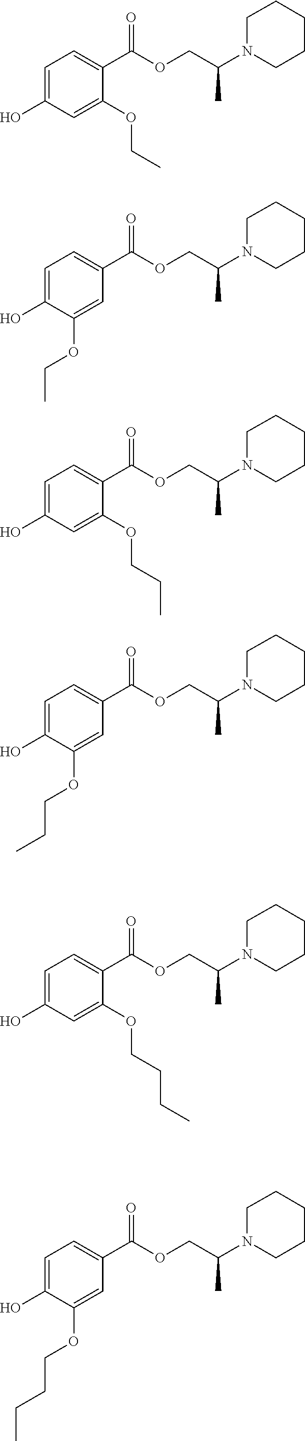 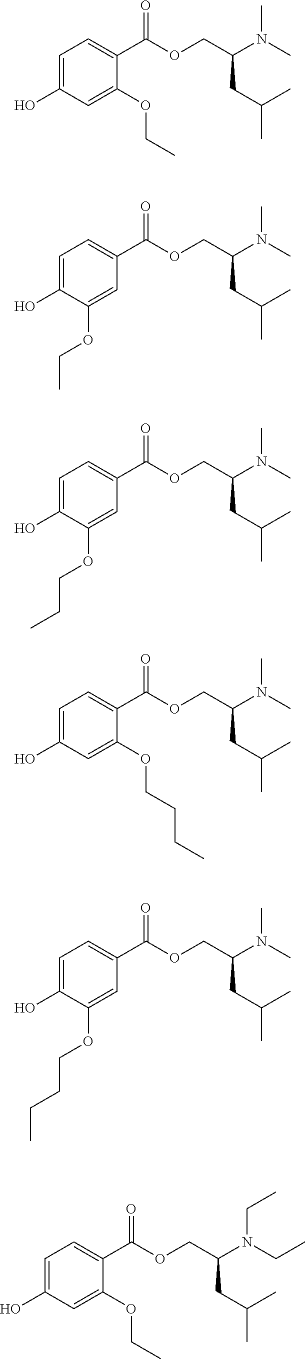 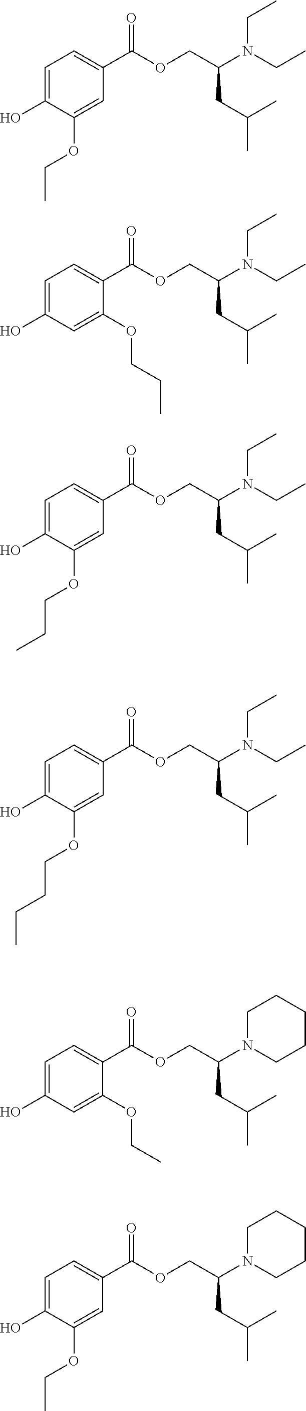 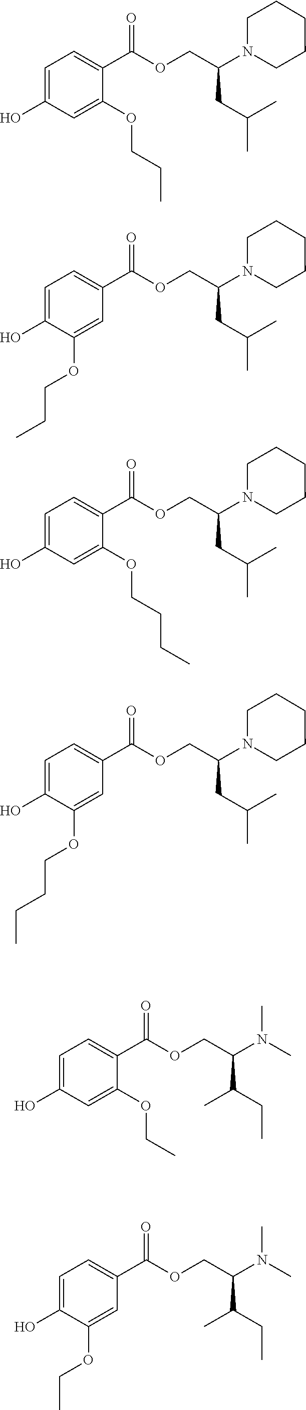 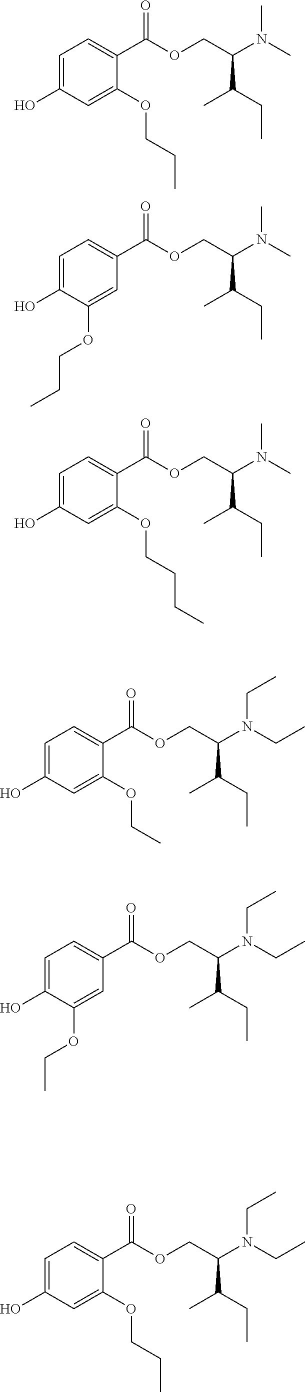 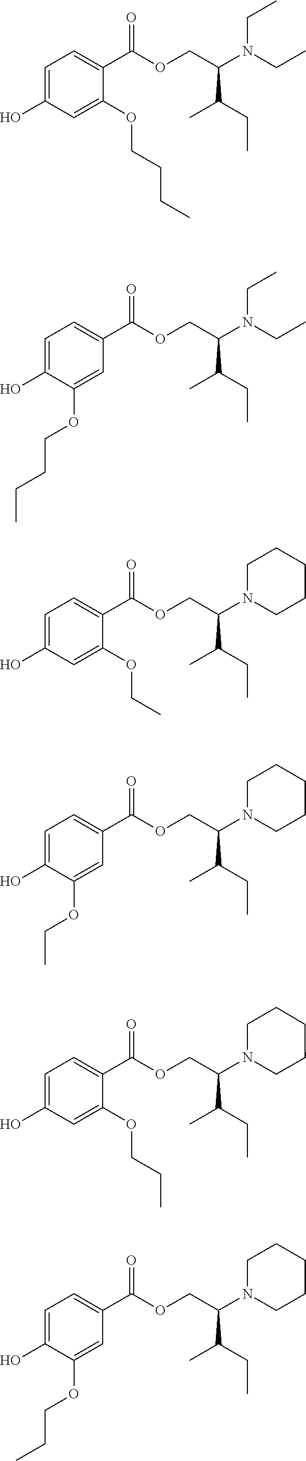 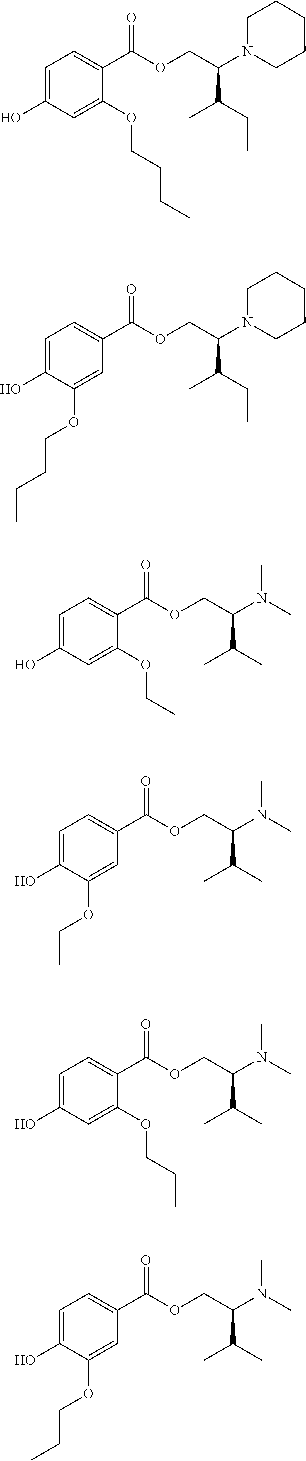 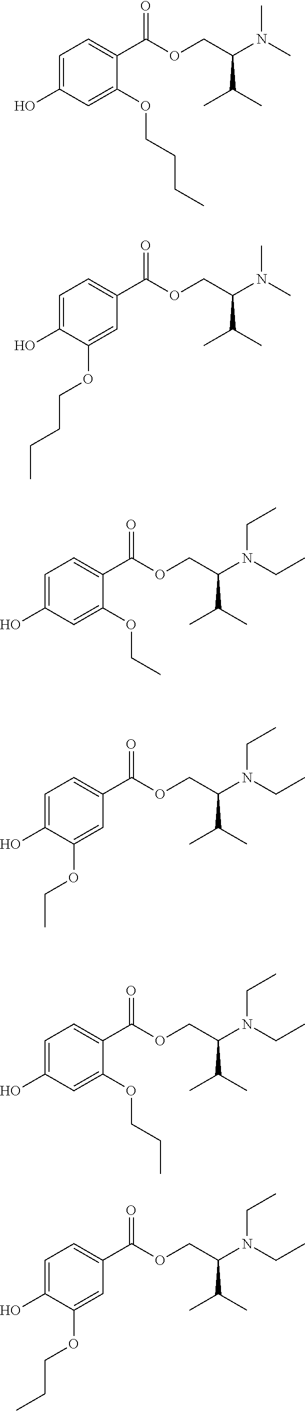 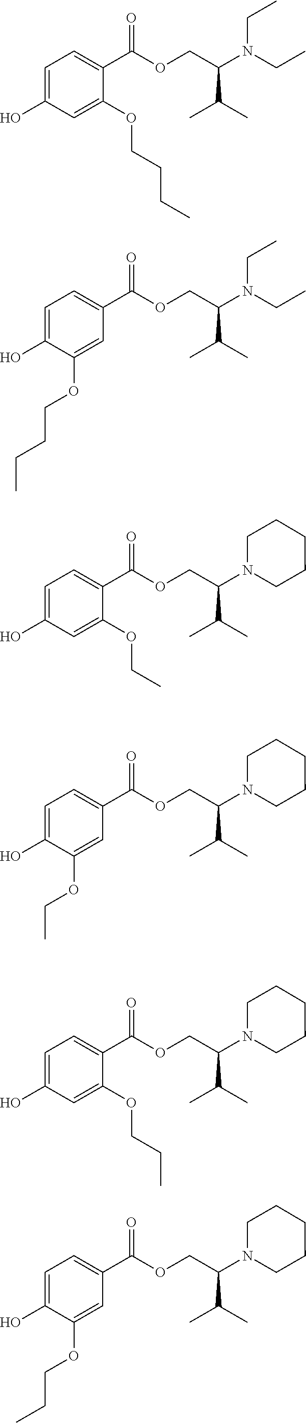 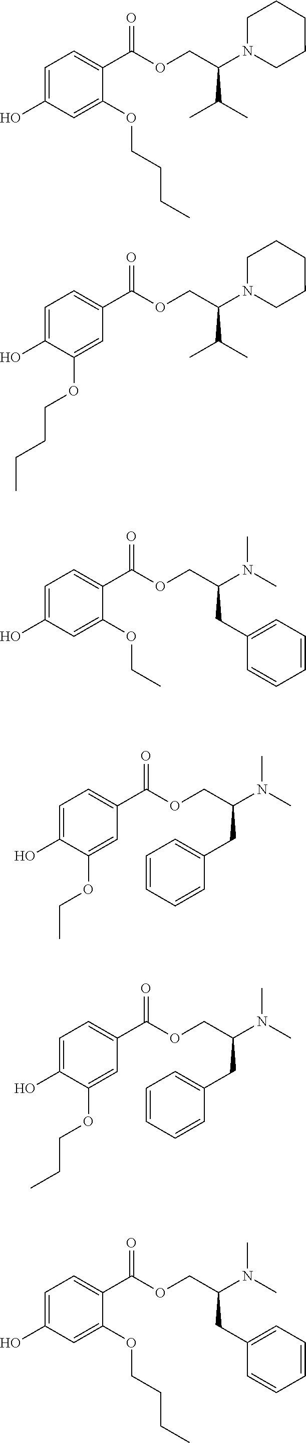 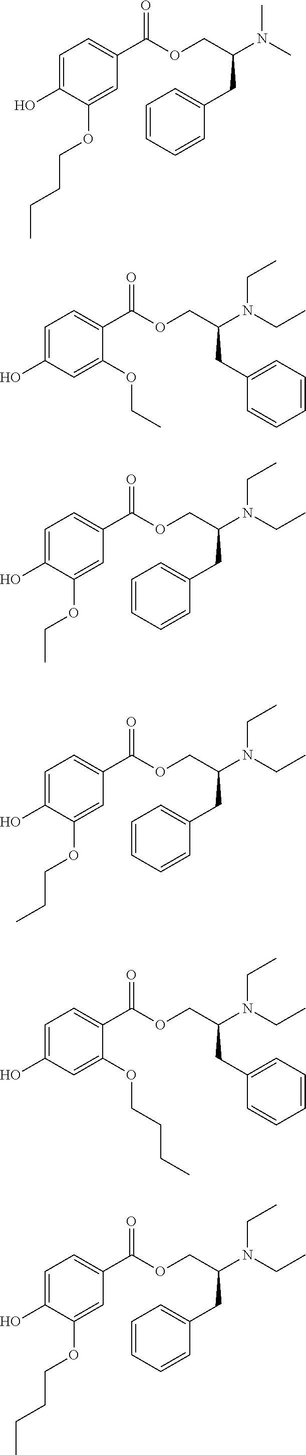 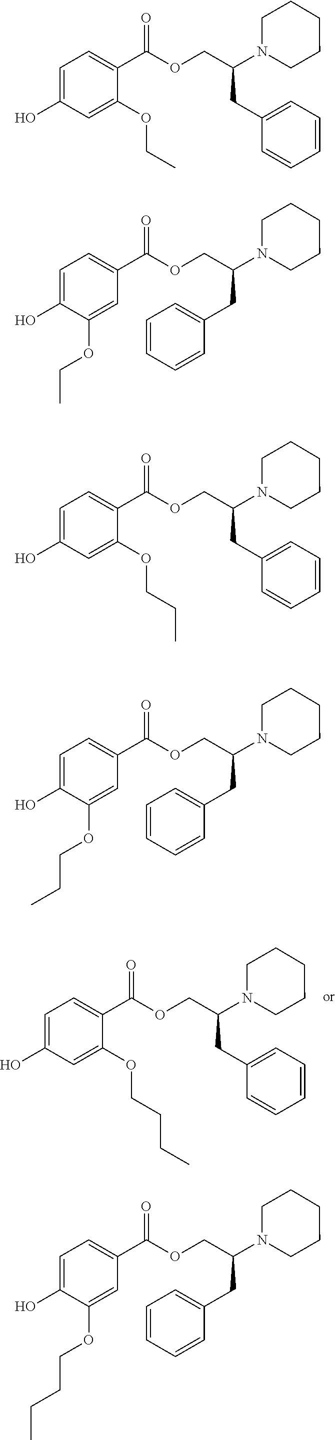
or a prodrug, salt, hydrate, solvate or N-oxide thereof.

2. A pharmaceutical composition comprising a compound as described in claim 1 and a pharmaceutically acceptable vehicle.

3. The composition of claim 2 which is formulated for injection and which further comprises an organic acid buffer system.

4. A method of inducing or maintaining local anesthesia in a patient comprising administering to the patient a compound as described in claim 1.

5. A method of inducing or maintaining local anesthesia in a patient comprising administering to the patient in need thereof the pharmaceutical composition of claim 2.

6. A method of treating or preventing pain in a patient comprising administering to the patient in need thereof a compound as described in claim 1.

7-9. (canceled)
